(19) United States
(12) Patent Application Publication
(10) Pub. No.: US 2016/0199613 A1
(43) Pub. Date: Jul. 14, 2016

(54) COMPRESSIBLE RESPIRATORY THERAPY HOSE AND COLLECTING ROD FOR STORAGE

(71) Applicant: INNOTECH LTD, Tel-Aviv (IL)

(72) Inventor: Noam HADAS, TEL AVIV (IL)

(21) Appl. No.: 14/909,991

(22) PCT Filed: Aug. 17, 2014
(86) PCT No.: PCT/IL2014/050739

§ 371 Date: Feb. 4, 2016

Related U.S. Application Data
(60) Provisional application No. 61/867,071, filed on Aug. 18, 2013.

(51) Int. Cl. A61M 16/08; A61M 16/06; A61M 16/00

(52) U.S. Cl. CPC A61M 16/0875; A61M 16/0816; A61M 16/0057; A61M 16/06

(57) ABSTRACT

A compressible respiratory gas hose system interconnects a breathing gas source 101 and a patient interface device. The hose system has an open position when a breathing gas flow from the gas source is conducted to the patient interface device and a closed position when the gas flow is blocked. The aforesaid hose system comprises: (a) a first connector connectable to the gas source; (b) a second connector connectable to the patient interface device; (c) a resilient helical support member, mechanically interconnecting the first and second connectors; (d) a flexible envelope accommodating the support member and providing gas interconnection between the first and second connectors; and (e) a rod reconfiguring the support member from the relaxed long position to the compressed storage position and vice versa.

### FIELD OF THE INVENTION

The present invention presents a respiratory hose for delivering a breathing air flow to a patient and, in particular, an improved hose that can be easily collapsed to a shorter length for the purpose of packing and/or storing, when not in use. The hose can be easily expanded to an operating size, when required.

### BACKGROUND

There is often a need to use a respiratory assisting device to support a patients' breathing. Such ventilation modalities are commonly known as Non Invasive Ventilation (NIV), Continuous Positive Air Pressure ventilation (CPAP), Bi-level Positive Air Pressure ventilation (BPAP) and others. The later modality is especially useful in treating patient for sleep apnea syndrome—obstructive, central or mixed, and other conditions such as congestive heart failure.

CPAP and BPAP therapy involve the placement of a mask on the patients' face over the nose or over the nose and the mouth, which is typically secured to the face using an elastic headgear or straps. It is critical that the mask does not move during therapy, since this will create leaks which may disrupt the ventilator pressure stability. The mask is normally connected by a large bore gas conduit, such as flexible hose, to a ventilator or other pressure generating device.

CPAP therapy for sleep apnea syndrome is a huge success world-wide, but while such treatment can resolve all symptoms and greatly benefits the patients, there are still drawbacks and difficulties that patients face when using these devices on their daily lives.

One such issue is using CPAP when traveling. The patient needs to pack the pressure generator, the hose, the mask and headgear. All these components of the therapeutic system are fragile, have odd shapes, are cumbersome to arrange and handle, and take up a lot of space to pack.

It is therefore clear that there is a need to develop smaller, more space efficient and more robust components in order to resolve at least some of the difficulties these patients encounter each day. Indeed, modern technology allows companies to offer progressively smaller and more robust pressure generators each year, however, the other components of the therapy system remained almost the same for years.

### SUMMARY OF THE INVENTION

The invention relates to a respiratory hose, intended to pass respiratory gases from a pressure generator such as a CPAP device, to a patient interface device (i.e. a nasal mask). It is the aim of this disclosure to detail how such a hose can be built so as to allow it to be compressed in length, from the relaxed length of about 200 cm, by a factor of 10 or more, in order to facilitate efficient packing for travel or storage. It is also the aim of this disclosure to describe how this hose can be manually and easily compressed from the full-length relaxed in-use configuration, to the compressed storage configuration. It is also the aim of this disclosure to describe a fixture designed to allow easy collection to the compressed configuration, and to hold it is this configuration until it needs to be expanded for use. The fixture can then easily release it, allowing the hose to expand to the full length without the users' intervention.

The hose assembly, described in the following, comprises a metal or very strong polymer helical support member with a typical length of 7 feet, intended to hold the inner lumen open under all normal use conditions. The support member is mechanically connected to a first standard conical connector on one end of the hose, and to a second, standard conical connector on the other end of the hose. These connectors are typically 22 mm or 15 mm in diameter.

The hose assembly, described in the following, further comprises an envelope in the shape of a long and narrow tube. The envelope is constructed of a very soft biocompatible plastic film, woven or non-woven sheet material of typically 0.1 mm to 0.3 mm thickness, with a diameter to match the required standard. The envelope is mechanically connected to the first and second connectors at both ends of the hose, forming a sealed internal lumen inside which the support member passes. The diameter of the envelope may vary periodically along the length of the hose, allowing it to fold over itself when the hose is compressed to the storage configuration. In the preferred embodiment there is no mechanical connection between the internal support member and the envelope wall, other than at the two ends of the hose.

The hose assembly further comprises a collection rod which can be inserted or threaded through the first or second connectors into the internal lumen of the hose, until it is fully inserted except for a stop plate assembled at first end of the collection rod. The collection rod further comprises a set of two retractable stops, located at the end, distal from the stop plate, which allow the loops of the support member to slide over the rod when pushed by the user in this direction, however the two retractable stops prevent the loops from sliding off the rod in the reverse direction under the elastic force of the support member when compressed. The retractable stops can be released upon actuating a dedicated lever or button on the collection rod base, in order to release the hose from its compressed configuration under the elastic force of the support member.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

It is hence one object of the invention to disclose compressible respiratory gas hose system for interconnecting a breathing gas source and a patient interface device. The aforesaid hose system has an open position when a breathing gas flow from said gas source is conducted to said patient interface device and a closed position when said gas flow is blocked. The hose system comprises: (a) a first connector connectable to said gas source; (b) a second connector connectable to said patient interface device; (c) a resilient helical support member, mechanically interconnecting said first and second connectors; (d) a flexible envelope accommodating said support member and providing gas interconnection between said first and second connectors; and (e) a rod reconfiguring said support member from said relaxed long position to said compressed storage position and vice versa.

Another object of the invention is to disclose the hose system with compressibility of said support member between said relaxed and compressed positions is characterized by a factor of at least 10.

A further object of the invention is to disclose the envelope and said support member which are only connected mechanically at said first and second connectors on both sides of said hose.

A further object of the invention is to disclose the collection rod comprising retractable stops guiding loops of said support member when said support member is reconfigured from said compressed position into said relaxed position and vice versa.

A further object of the invention is to disclose the collection rod comprising a release button, retracting said retractable stop configured to assist reconfiguring said support member into said relaxed position and vice versa.

A further object of the invention is to disclose the collection rod having a tip configured for manually coiling of said loops of said support member over said rod body.

A further object of the invention is to disclose the collection rod having a body diameter 0.2 to 5 mm smaller than an inside loop diameter of said support member.

A further object of the invention is to disclose the support member having an outside loop diameter 0.5 to 5 mm smaller than the inside diameter of said envelope.

A further object of the invention is to disclose the support member which is a helical compression spring having an outside diameter of 10 to 40 mm, a wire diameter of 0.3 to 1.5 mm, and a coil step distance of 5 to 30 mm.

A further object of the invention is to disclose the support member length in said relaxed position which is longer than the total length of said envelope by 5—30 mm; said support member applies stretching forces said envelope to compensate material aging and temperature effects of said support member and said envelope.

A further object of the invention is to disclose the envelope made of a material selected from the group consisting of a vinyl film, a polyethylene film, a polyurethane film, a woven sheet material, a non-woven sheet material and any combination thereof.

A further object of the invention is to disclose the film or material treated by at least one of the following technology: lamination and impregnation.

A further object of the invention is to disclose the envelope material having a thickness ranging between 0.05 to 0.5 mm.

A further object of the invention is to disclose the first and second connectors which can be disassembled by said user, allowing replacement of either said support member and/or said envelop, as needed.

A further object of the invention is to disclose the support member having a variable step pitch, such that support member locations being under higher compression load are characterized by shorter helix loop pitch while member locations being under less compression load have a longer helix loop pitch.

A further object of the invention is to disclose the loops of said support member being in the compressed, short configuration which are brought to the shortest possible distance between them, and no part of said envelop is trapped between adjacent loops.

A further object of the invention is to disclose an air pressure ventilation system comprising: (a) an air flow generator; (b) a face mask; and (c) an air delivery tube configured for interconnecting said air flow generator and said face mask.

It is a core purpose of the invention to provide the wherein said air delivery tube is convertible from a collapsed storage position into a flexibly extended operation position and vice versa.

A further object of the invention is to disclose the air delivery tube comprising: (a) a tubular flexible envelope conducting air flow from said an air flow generator to said face mask; and (b) a resilient helical support member having plurality of coils, accommodated within said tubular flexible envelope, configured to be convertible from an extended position into a compressed position and vice versa.

A further object of the invention is to disclose the air delivery tube comprising two terminal portions. Each terminal portion further comprises: (a) a fixating part, delivery tube mechanically connected to said envelope and said helical support member; and (b) a connection part configured for connecting said terminal portion to said air flow generator and to said face mask, such that a fluid connection is established.

A further object of the invention is to disclose the system comprising a collection rod reconfiguring said attachable air delivery tube from said relaxed position into said compressed position and vice versa.

A further object of the invention is to disclose the collection rod having a first end and a second end. The collection rod comprises: (a) a tip at a first end of said collection rod configured to be screwed into said air delivery tube; (b) at least one retractable first protruding stopper at said first end; (c) at least one second protruding stopper at a second end of said collection rod; and (d) a release button, at said second end of said collection rod, configured to release said at least one retractable protruding stopper, such that said air delivery tube can be released from said collection rod.

A further object of the invention is to disclose the retractable stoppers rotatable around axes and to be spaced apart from each other, by a compression spring mounted between said stoppers.

A further object of the invention is to disclose the collection rod comprising a moveable slider inserted in a hollow volume inside said collection rod.

A further object of the invention is to disclose the collection rod further comprises a V-shaped slot connected by two engaging pins to said two retractable stoppers, configured to prevent said two retractable stoppers from further protruding;

A further object of the invention is to disclose the two retractable stoppers configured to be pressed into said hollow volume when pushed by said threaded connectors connection parts, fixating parts or said coils of said resilient helical support member.

A further object of the invention is to disclose the two retractable stoppers configured to be snapped back into said protruded position, for holding said coils, said connection parts and/or said fixating parts, on the steep faces of said two retractable stoppers.

### BRIEF DESCRIPTION OF THE DRAWINGS

FIG. 1 is an overview of the compressible respiratory gas hose system and the collection rod;

FIG. 2 is close-up view of one end of the hose, showing the ventilator connector, support member and envelope, in the extended configuration;

FIG. 3 is an overview of the collection rod;

FIG. 4 is a view of the entire hose in the collapsed configuration threaded onto the collection rod; and

FIG. 5 is a close up section view of the internal details of the folded spring loaded stops at the end of the collection rod.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body.

As employed herein, the statement that two or more parts or components “engage” one another shall mean that the parts exert a force against one another either directly or through one or more intermediate parts or components.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Since the aspect ratio of the hose in both relaxed and compressed configurations is very high, it is impossible to draw the device or parts of the device to scale. The length dimension is therefore drawn cut with two wavy lines with a small space between them, and this should be understood as including the entire undrawn length of the device or part, which does not have any new details not showing on the drawn parts. Lines which end with several small dashed lines should be understood as continuing along the length of the device or part.

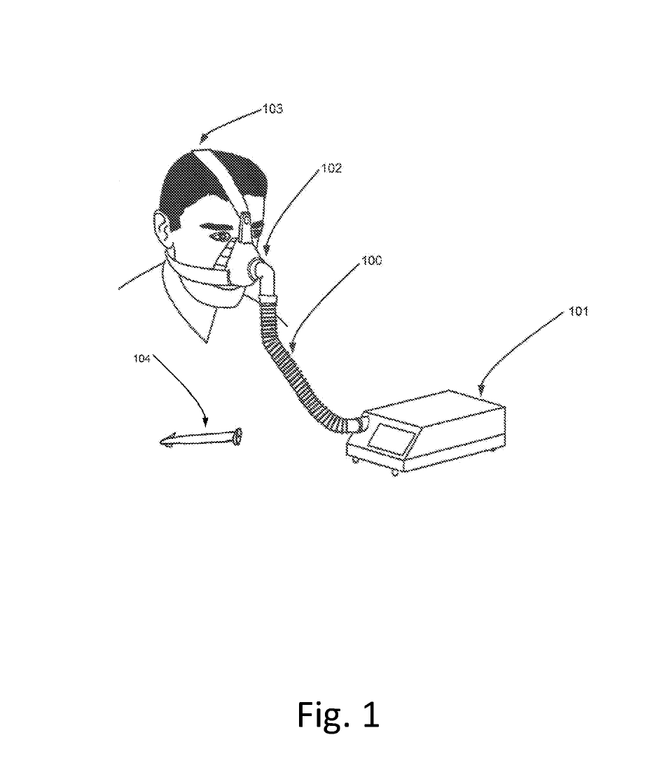

A general view of the compressible respiratory therapy hose 100 in accordance with a first embodiment of the present invention is depicted generally in FIG. 1. Hose 100 is a flexible, tubular element that is designed and intended to be connected between a source of pressurized breathable gas 101 and an respiratory interface device 102 held securely on the patient's face, usually using an elastic harness of set of straps 103. The source of pressurized breathable gas 101 may be selected from a group consisting of CPAP, BPAP, VPAP, pressure support ventilator and any of a variety of similar devices intended for respiratory support, by providing higher than atmospheric pressure air or other breathable gas mixture to a patient.

The compressible respiratory therapy hose 100 in accordance with a first embodiment of the present invention, as depicted generally in FIG. 1, is presented in the first relaxed, full length configuration. In this configuration it is of a length considered be the optimal length for a hose that supplies breathable air from the pressure-support device positioned near the bed, and the interface on the face of a patient lying in the bed.

When the respiratory therapy hose 100 is in its relaxed, full length configuration, as is depicted generally in FIG. 1, the internal support member 205 is expanded, such that the loops of the helix provide sufficient kink and compression resistance, while at the same time offering excellent flexibility, thereby allowing the patient full freedom of motion, without generating significant pull and/or rotation and/or sliding forces on the patient interface.

The envelope 203 surrounding the support member 205 inflates to its maximum diameter, creating a long lumen through which respiratory gases are passed from the pressure support device to the patient. The enlarged diameter of the inflated envelope 203 form a path that presents a very low resistance to gas flow, as the patient breathes, insures the accuracy of pressure provided to the patient and is controlled by the pressure support device on the distal (from the patient) end of the hose.

While the cross section of the support member 205 and the envelope 203 as presented herein is circular, the design is not intended to be limited to this shape but can be realized following shapes such as elliptical shapes, oval shapes, and the like without limitation.

As can be easily understood from FIG. 1, the hose is connected to a pressure support device 101 on the first end, and to a patient interface device 102 on the second end. The connectors must therefore be compatible with these types of devices found on the market.

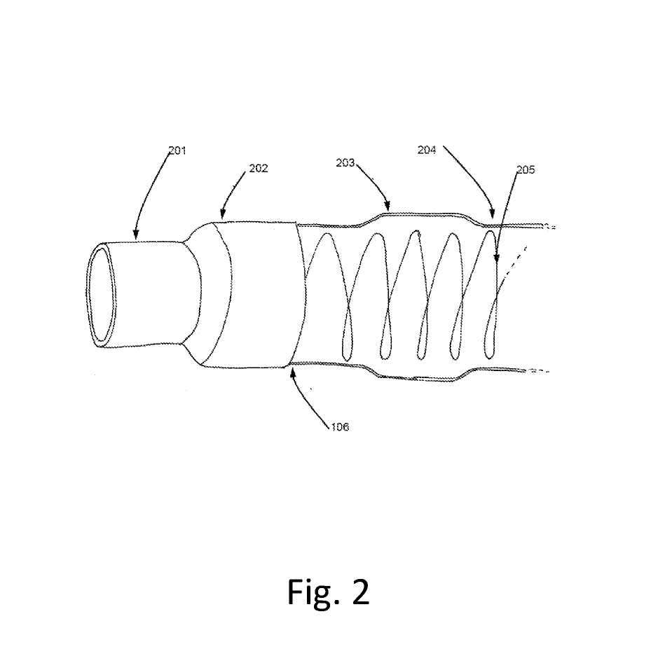

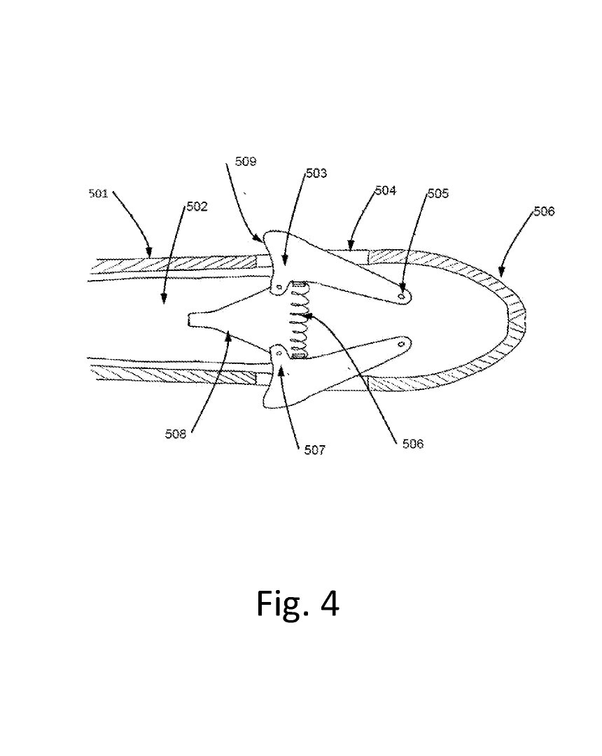

The hose comprises several components which are more clearly seen in FIG. 2., depicting a close up-view of one end of the hose. These are the connectors (similar connectors, one on each end), the support member 205 and the envelope 203. In FIGS. 2 and 4, the envelope material is considered transparent, allowing clear view of the loops of the internal support member.

The hose 100 comprises a support member 205, which is of helix shape, similar in design to standard compression helical springs. In one embodiment, the support member is produced from a spring quality stainless steel wire with a diameter in the range of 0.5-1.5 mm, formed into a circular helix with an internal diameter in the range of 10-30 mm, and with a distance between loops of the helix in the range of 0.5-3 cm. the support member 205 spans the entire length of the hose 100 and is secured at both ends to the connectors at the ends of the hose 100.

It must be understood that the typical dimensions presented in the previous paragraph are brought as for guidance only, and other possible embodiments are possible. It is important to present one special deviation from this description, where, in order to minimize weight and cost of the support member 205, the distance between the helix loops changes along the length of the hose 100. The loops are more closely spaced where significant compression forces are expected to be present on the hose 100, such as at the center part of the hose which may be caught under the users' body as they roll in bed, and are more widely spaced in other parts of the hose, where such forces are less likely to be present, such as close to the ventilator connector, or the patient interface connector.

Hose 100 further comprises two connectors, one of the first end and one of the second end. These connectors must be detachable connectors designed to be compatible with the industry standard and regulation for such connectors, as detailed in ISO 14971. Of course, other types of connectors may be fitted to the compressible center part of the hose, as may fit the application while still following the teaching in this disclosure in all other design aspects. As can be understood from FIG. 2, both support member 205, and envelope 203, are secured to the connectors on both first and second ends of the hose assembly. Each connector may be a single piece design, having the correct shape and dimensions to securely connect to either the pressure support device output connector, or the patient interface input connector, on one end, and to the support member and envelope on the other, either through a mechanical connection or by molding of the connector body over the ends of the support member and envelope.

In an alternative design, the connectors on the first and second ends of the hose 100 comprise two separate parts 201 and 202, which can be assembled and disassembled in production and possibly also by the final user at the place of therapy delivery, or by a technician at a service location. In this embodiment, part 202 can be screwed on or locked in any other fashion over part 201, while locking support member 205 and envelope material in a specially formed groove or slot which forms between the mating faces of the part as they match. This design allows the replacement of either the support member 205 and/or the envelope 203 at any time after production, as a mean for servicing or repairing the hose 100 when needed. It is important to note that in whatever embodiment of the connectors, the envelope 203 forms an air-tight connection with the connector body.

The inner edges of the connector on the side distal to the external device connection 206 can be made rounded, in order to minimize wear to the envelope 203 material as it bends over these edges as the hose 100 moves in use.

Hose 100 further comprises an envelope 203, forming the hollow tubular path for transferring higher than ambient pressure respiratory gases from the pressure support device 101 to the patient interface device 102. This envelope 203 is made of a very soft and foldable non-stretchable or slightly elastic film. or a woven or non-woven sheet material, with a thickness in the range of 0.05 m-1 mm. Possible embodiments may include films selected from a group comprising films made of PVC or other vinyl materials, PE, PU, films manufactured from other polymer types and woven and non-woven materials such as Tyvek® either with or without an additional laminated or applied polymeric layer. The film selected for making the envelope must be very flexible and compressible, be airtight to a degree sufficient to comply with the maximum allowed leak from the hose, be medical grade, and be of a type that does not shed fibers or particles which may be caught in the flow of air in the internal lumen and be breathed by the patient.

While in the extended, full length configuration, the envelope 203 forms a sealed tube like shape spanning from the connector on the first end coupled to the respiratory support device 101, to the connector on the second end of the hose 100, coupled to the patient interface device 102. The higher than atmospheric gas pressure inside the sealed volume of the envelope 203 inflates it to the maximum diameter, creating a low flow resistance path from the ventilator to the patient. The support member 205 inside the lumen thus formed prevents the envelope 203 from blocking, restricting flow or increasing flow resistance due to kinking or compression forces as the patient moves to different postures, or even lies on a section of the hose 100. The flexibility of the envelope 203 does not hinder free motion of the user, without generating significant pull and/or rotation and/or sliding forces on the patient interface.

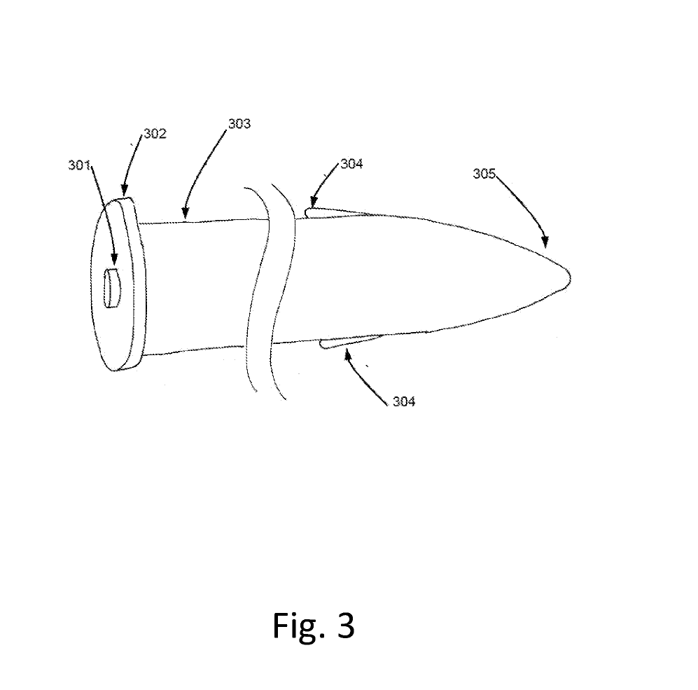

The compressible respiratory therapy hose system 100 in accordance with a first embodiment of the present invention as depicted generally in FIG. 1, also comprises a collection rod 104. The rod 104 is intended to enable the user of the hose 100 to quickly and effortlessly change the configuration of the hose 100 from the relaxed, full length configuration, to the second compressed, storage configuration and vice versa. It is also intended to hold the hose 100 in the compressed, storage configuration until released by the user. It is also intended to guard the hose 100 from deformations and breakage while in the compressed configuration, from forces and efforts which may be inflicted on the hose 100 during storage, packing and shipment. A more detailed view of the collection rod 104 appears in FIG. 3.

The construction and use of the collection rod 104 can be better understood by looking at FIG. 3. The rod body 303 is a tubular element with a diameter slightly smaller than the internal diameter of the support member 205. The body diameter is also slightly smaller than the diameter of the lumen in the connectors on the first and second ends of the hose 100. It is therefore possible to slide first one connector over the rod 104, and continue to slide the support members' 205 coils over the rod 104.

The connector first threaded over the rod 104 this way is pulled along the length of the rod 104 until it is stopped from falling off the other end of the rod 104 by stop plate 302. The coils of the support member 205 are then pulled over the rod 104, and the smooth, rounded, elongated tip 305 of the collection rod 104 enable smooth threading through the loops of the support member 205. As each new loop is threaded on the rod 104, it passes over the two retractable stops 304 assembled near the distal (from the stop plate 302) end of the rod 104. The retractable stops 304 are movable in such a way that they can be pushed into the body of the rod 104, and are spring loaded to stay in the protruding position until pressed inside. As each loop passes over the stops 304, they move into the body to allow the loop to slide over the rods body 303, but they immediately snap back into the protruding position again, preventing the coils of the support member 205 from sliding back up the rod 104. It is therefore very easy for the user to collect more and more of the hose 100 by holding a section of the hose 100, and sliding coils of the support member 205 over the rod 104 until reaching the connector on the second end of the hose.

Both connectors comprise grooves on the inner surface of the lumen of the connector, close to the end that connects to the external device, either the pressure source 101 or patient interface 102. As the second connector is threaded over the rod 104, the retractable stops 304 snap and lock into these grooves, thus holding the connector securely in place. The hose 100 is now totally compressed over the hose 100, as is depicted in FIG. 4.

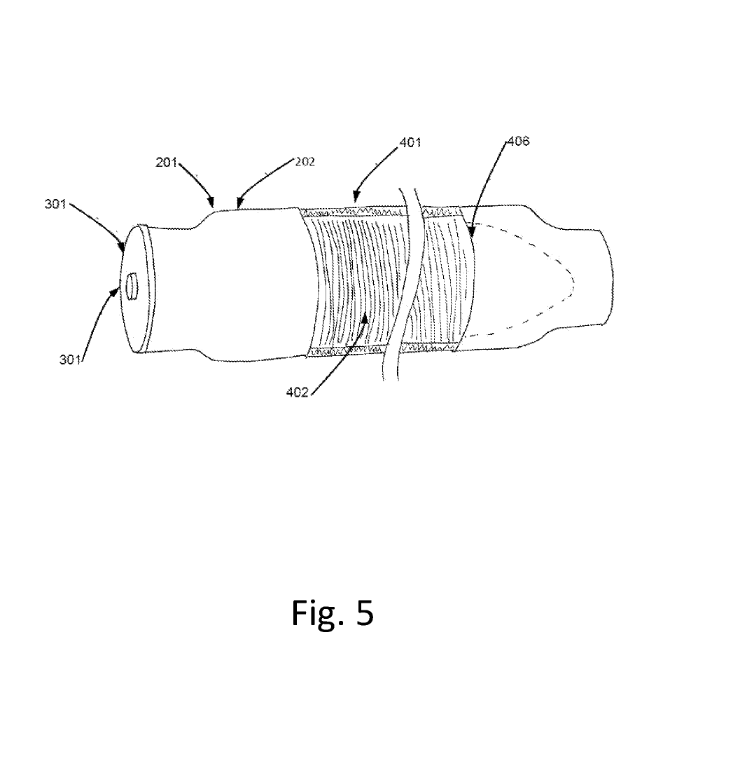

In FIG. 4. the hose 100 is presented in the fully compressed storage configuration over the collection rod 104. The loops of the support member 205 are pressed closer and closer as the hose 100 is collected, until they actually touch when the hose 100 is totally collapsed setting the minimum possible length of the hose 100 in the compressed configuration. Note that the envelope 203 material folds into many very dense folds and waves as the hose 100 length decreases by a factor of 10 or more. Since the envelope 203 material is not mechanically connected to the loops of the support member 205 at any point other than at the two connectors, it forms into a sheath of folded, compressed material 401 around the compressed support member 402, but at no point does the material get between the loops of the support member 205 as they become progressively closer. Instead, it collapses over itself in a separate cylinder 401. Since the loops of the support member 205 are compressed until they can touch, the hose 100 can be compressed to the minimum possible length as dictated by the ration of the distance between loops of the support member 205 when relaxed, to the diameter of the wire from which it is manufactured. Using typical values of 2 cm as the distance between the loops, and 1 mm as the diameter of the wire, it can be calculated that a 200 cm long hose 100 can be compressed to a total length of just 10 cm, plus the length of the two connectors, or typically about 7% of the relaxed length. The dashed line inside the connector on the right represents the approximate location of the tip 506 of the collection rod (506, FIG. 5).

Pushing the release button 301 on the stop plate forces the two retractable stops to be retract into the rods' body. The stops thereby release the connector and the loops of the support member 205. The hose 100 then spreads immediately and automatically to its full length under the elastic forces of the compressed support member 205, making the hose 100 ready for use.

FIG. 5. depict a cross section of the collection rod 104, presenting the detailed design of the retractable stops 304 and their operation. Each stop 304 is preferably made of rigid plastic 503, and is free to rotate on axis 505. The two stops 304 are pushed away from each other by the force of compression spring 506 locked between them. A moveable slider 502 is inserted in the hollow volume of the collection rods' body, and has a V shaped slot 508 at its end engaging pins 507 on the stops. The pins 507 press against the slot 508 internal walls preventing the stops from opening any further, but the stops can and will move into the hollow space in the rod if pushed from the outside, as happens when sliding the loops of support member 205 over them, or sliding the connector over them. As soon as these forces disappear, the stops 304 snap back into the protruding position, supporting the loops or connector on the steep faces 509 which are at an angle that create forces that support the protruding position of the stops 304.

Slider 502 runs along the entire length of the collection rod 104, and a button shaped cylinder fashioned on its other end protrudes through a hole in the stop plate (302, FIG. 4) to present to the user as the release button (301, FIG. 4). When pressed, the slider 502 moves to the right side (of the figure), and slot 508 forces pins 507 to move and pull the stops into the body of the rod 100, releasing the hose. As soon as the button is released, the slider 502 moves back to the original position under the force of spring 505 pushing on the pins 507.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A compressible respiratory gas hose system 100 for interconnecting a breathing gas source 101 and a patient interface device 102, said hose system having an open position when a breathing gas flow from said gas 101 source is conducted to said patient interface device and a closed position when said gas flow is blocked, said hose system comprising:
a. a first connector connectable to said gas source;
b. a second connector connectable to said patient interface device;
c. a resilient helical support member, mechanically interconnecting said first and second connectors;
d. a flexible envelope accommodating said support member and providing gas interconnection between said first and second connectors; and
e. a rod reconfiguring said support member from said relaxed long position to said compressed storage position and vice versa.

2. The hose system of claim 1, wherein compressibility of said support member between said relaxed and compressed positions is characterized by a factor of at least 10.

3. The hose system of claim 1, wherein said envelope and said support member are only connected mechanically at said first and second connectors on both sides of said hose.

4. The hose system of claim 2, wherein said collection rod comprises retractable stops guiding loops of said support member when said support member is reconfigured from said compressed position into said relaxed position and vice versa.

5. The hose system of claim 3, wherein collection rod comprises a release button, retracting said retractable stop configured to assist reconfiguring said support member into said relaxed position and vice versa.

6. The hose system of claim 2, wherein said collection rod has a tip configured for manually coiling of said loops of said support member over said rod body.

7. The hose system of claim 3, wherein said collection rod has a body diameter 0.2 to 5 mm smaller than an inside loop diameter of said support member.

8. The hose system of claim 3, where said support member has an outside loop diameter 0.5 to 5 mm smaller than the inside diameter of said envelope.

9. The hose system of claim 3, wherein said support member is a helical compression spring having an outside diameter of 10 to 40 mm, a wire diameter of 0.3 to 1.5 mm, and a coil step distance of 5 to 30 mm.

10. The hose system of claim 3, wherein said support member length in said relaxed position is longer than the total length of said envelope by 5—30 mm; said support member applies stretching forces said envelope to compensate material aging and temperature effects of said support member and said envelope.

11. The hose system of claim 3, wherein said envelope is made of a material selected from the group consisting of a vinyl film, a polyethylene film, a polyurethane film, a woven sheet material, a non-woven sheet material and any combination thereof.

12. The hose system of claim 10, wherein said film or material is treated by at least one of the following technology: lamination and impregnation.

13. The hose system of claim 10, wherein said envelope material has a thickness ranging between 0.05 to 0.5 mm.

14. The hose system of claim 3, wherein said first and second connectors can be disassembled by said user, allowing replacement of either said support member and/or said envelop, as needed.

15. The hose system of claim 3, wherein said support member has a variable step pitch, such that support member locations being under higher compression load are characterized by shorter helix loop pitch while member locations being under less compression load have a longer helix loop pitch.

16. The hose system of claim 3, wherein while in said compressed, short configuration, said loops of said support member are brought to the shortest possible distance between them, and no part of said envelop is trapped between adjacent loops.

17. An air pressure ventilation system 10 comprising:
a. an air flow generator 101;
b. a face mask 102; and
c. an air delivery tube 100, configured for interconnecting said air flow generator 101 and said face mask 102;
wherein said air delivery tube 100 is convertible from a collapsed storage position into a flexibly extended operation position and vice versa.

18. The system 10 according to claim 14, wherein said air delivery tube 100 comprising:
a. a tubular flexible envelope 203, conducting air flow from said an air flow generator 101 to said face mask 102; and
b. a resilient helical support member 205 having plurality of coils 401, accommodated within said tubular flexible envelope 203, configured to be convertible from an extended position into a compressed position and vice versa.

19. The system 10 according to claim 14, wherein said air delivery tube 100 comprises two terminal portions; each terminal portion further comprises:
a. a fixating part 202, delivery tube 100 mechanically connected to said envelope 203 and said helical support member 205; and
b. a connection part 201, configured for connecting said terminal portion to said air flow generator 101, and to said face mask 102, such that a fluid connection is established.

20. The system 10 according to claim 14, wherein said system 10 further comprises a collection rod 104 reconfiguring said attachable air delivery tube 100 from said relaxed position into said compressed position and vice versa.

21. The system 10 according to claim 16, wherein said collection rod 104 has a first end and a second end; said collection rod comprises:
a. a tip 305 at a first end of said collection rod 104 configured to be screwed into said air delivery tube 100;
b. at least one retractable first protruding stopper 304 at said first end;
c. at least one second protruding stopper at a second end of said collection rod 104; and
d. a release button 301, at said second end of said collection rod 104, configured to release said at least one retractable protruding stopper 304, such that said air delivery tube 100 can be released from said collection rod 104.

22. The system 10 according to claim 17, wherein said retractable stoppers 304 are rotatable around axes 505 and to be spaced apart from each other, by a compression spring 506 mounted between said stoppers;

23. The system 10 according to claim 17, wherein said collection rod 104 further comprises a moveable slider 502 inserted in a hollow volume inside said collection rod 104.

24. The system 10 according to claim 17, wherein said collection rod 104 further comprises a V-shaped slot 508 connected by two engaging pins 507 to said two retractable stoppers 304, configured to prevent said two retractable stoppers 304 from further protruding.

25. The system 10 according to claim 17, wherein said two retractable stoppers 304 are configured to be pressed into said hollow volume when pushed by said threaded connectors connection parts 201, fixating parts 202 or said coils 401 of said resilient helical support member 205.

26. The system 10 according to claim 17, wherein said two retractable stoppers 304 are configured to be snapped back into said protruded position, for holding said coils 401, said connection parts 201 and/or said fixating parts 202, on the steep faces 509 of said two retractable stoppers 304.
